SEQUENCE LISTING (32 sequences)

SEQ ID NO: 1 - protein, 10 aa, Artificial Sequence
  BB0500-1 Hinge Sequences
CKTHTCPPCP

SEQ ID NO: 2 - protein, 11 aa, Artificial Sequence
  BB0500-2&BB0500-6 Hinge Sequences
DKTCHTCPPCP

SEQ ID NO: 3 - protein, 12 aa, Artificial Sequence
  BB0500-3 Hinge Sequences
ERKSCVECPPCP

SEQ ID NO: 4 - protein, 15 aa, Artificial Sequence
  BB0500-5&BB0500-2a&BB0500-7 Hinge Sequences
EPKSCDKTHTCPPCP

SEQ ID NO: 5 - protein, 15 aa, Artificial Sequence
  BB0500-2b&BB0500-7a Hinge Sequences
EPKSDCKTHTCPPCP

SEQ ID NO: 6 - protein, 15 aa, Artificial Sequence
  BB0500-2c&BB0500-6a&BB0500-7b Hinge Sequences
EPKSDKCTHTCPPCP

SEQ ID NO: 7 - protein, 17 aa, Artificial Sequence
  BB0500-2d Hinge Sequences
EPKSDCKTHTVECPPCP

SEQ ID NO: 8 - protein, 15 aa, Artificial Sequence
  BB0500-2e Hinge Sequences
EPKSDCKTVECPPCP

SEQ ID NO: 9 - protein, 17 aa, Artificial Sequence
  BB0500-2f Hinge Sequences
EPKSDKCTHTVECPPCP

SEQ ID NO: 10 - protein, 18 aa, Artificial Sequence
  BB0500-2g&BB0801 Hinge Sequences
EPPKSCDKTHTVECPPCP

SEQ ID NO: 11 - protein, 18 aa, Artificial Sequence
  BB0500-2n Hinge Sequences
EPPKSDCKTHTVECPPCP

SEQ ID NO: 12 - protein, 19 aa, Artificial Sequence
  BB0802 Hinge Sequences
EPPPKSCDKTHTVECPPCP

SEQ ID NO: 13 - protein, 20 aa, Artificial Sequence
  BB0803 Hinge Sequences
EPPPPKSCDKTHTVECPPCP

SEQ ID NO: 14 - protein, 445 aa, Artificial Sequence
  BB0500-3 heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 15 - protein, 449 aa, Artificial Sequence
  BB0500-5 heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 449 aa, Artificial Sequence
  BB0500-2a heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 449 aa, Artificial Sequence
  BB0500-2b heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSDCKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 449 aa, Artificial Sequence
  BB0500-6a heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSDDTAIYYCARALTYYDYEFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSDKCTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 450 aa, Artificial Sequence
  BB0500-2f heavy chain
QVQLQESGPGLVKPSETLSLTCTVSGGSVSSGDYYWTWIRQSPGKGLEWIGHIYYSGNANYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSDKCTHTVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 20 - protein, 451 aa, Artificial Sequence
  BB0500-2g heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVAGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPPKSCDKTHTVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 21 - protein, 452 aa, Artificial Sequence
  BB0500-2n heavy chain
QVQLQESGPGLVKPSETLSLTCTVSGGSVSSGDYYWTWIRQSPGKGLEWIGHIYYSGNANYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPPKSDCKTHTVECPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 456 aa, Artificial Sequence
  BB0802 heavy chain
EVQLLESGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPPPKSCDKTHTVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 214 aa, Artificial Sequence
  light chain
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, Artificial Sequence
  light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 214 aa, Artificial Sequence
  light chain
DIQMTQSPSSLSASVGDRVTITCQASQDISNYLNWYQQKPGKAPKLLIYAASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, Artificial Sequence
  light chain
DIQMTQSPSSLSASVGDRVTITCQASQDISNALNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 213 aa, Artificial Sequence
  light chain
QYELTQPPSVSVYPGQTASITCSGDQLGSKFVSWYQQRPGQSPVLVMYKDKRRPSEIPERFSGSNSGNTATLTISGTQAIDEADYYCQAWDSSTYVFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 18 aa, Artificial Sequence
  BR0301&BR0302 Hinge Sequences
EPPKSDCKTKTVECPPCP

SEQ ID NO: 29 - protein, 452 aa, Artificial Sequence
  BR0301 heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPPKSDCKTKTVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 453 aa, Artificial Sequence
  BR0302 heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPPKSDCKTKTVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, Artificial Sequence
  BR0301 light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, Artificial Sequence
  BR0302 light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0075154 A1
(43) Pub. Date: Mar. 7, 2024

(54) ENGINEERED ANTIBODY, ANTIBODY-DRUG CONJUGATE, AND USE THEREOF

(71) Applicant: BLISS BIOPHARMACEUTICAL (HANGZHOU) CO., LTD., Hangzhou, Zhejiang (CN)

(72) Inventors: Bing XIA, Hangzhou, Zhejiang (CN); Yuhong ZHOU, Hangzhou, Zhejiang (CN); Ziping WEI, Hangzhou, Zhejiang (CN); Jianfeng YANG, Hangzhou, Zhejiang (CN)

(21) Appl. No.: 18/253,677

(22) PCT Filed: Nov. 19, 2021
(86) PCT No.: PCT/CN2021/131757

§ 371 Date: May 19, 2023

Related U.S. Application Data
(63) Continuation-in-part of application No. PCT/CN2020/130409, filed on Nov. 20, 2020.

(51) Int. Cl. A61K 47/68; C07K 16/28

(52) U.S. Cl. CPC A61K 47/68031; A61K 47/6849; A61K 47/6889; C07K 16/2863; C07K 2317/522; C07K 2317/524; C07K 2317/526; C07K 2317/53

(57) ABSTRACT

An isolated IgG antibody includes two identical heavy chains each having a hinge region with an amino acid sequence containing an additional cysteine upstream of the two cysteines in the CPPCP sequence of a native IgG hinge region, and a CH1 domain containing a cysteine at the position of 142 according to the IMGT numbering scheme. ADCs based on the antibody having this architecture are also provided.

### CROSS REFERENCE TO RELATED APPLICATIONS

This application claims priority to International Application No. PCT/CN2020/130409 filed Nov. 20, 2020, the disclosure of which is incorporated by reference herein in their entireties.

### BACKGROUND

Antibodies are key immune molecules acting against foreign pathogens. The development of monoclonal antibody (mAb) technology resulted in widespread use of monoclonal antibodies in research, diagnosis and treatment of diseases. The therapeutic use of first-generation mAb (mostly monospecific, bivalent mAb) achieved success in the treatment of a variety of diseases, including cancer, autoimmune, and infectious diseases. However, many diseases, such as solid tumors, have been shown to be quite resistant to antibody-based therapies.

Antibody Drug Conjugates (ADCs) are mAbs chemically linked to active drugs, and therefore, have both the specific targeting of mAbs and the cancer-killing ability of cytotoxic drugs. The ability to select specific mAbs-drug combination and advances in the linking the mAbs and drugs provide new possibilities to target cancers while minimizing exposure of healthy tissue. By 2019, a total of seven ADCs have been approved by the FDA, including: ado-trastuzumab emtansine (Kadcyla™), brentuximab vedotin (Adcetris™) inotuzumab ozogamicin (Besponsa™) gemtuzumab ozogamicin (Mylotarg™), polatuzumab vedotin-piiq (Polivy™), Enfortumab vedotin (Padcev™) and Trastuzumab deruxtecan (Enhertu™). In addition to the seven ADC drugs that have been approved for marketing, a large number of ADCs are currently under clinical development.

Conventional approaches of attaching a drug to an antibody generally lead to a heterogeneous mixture of ADCs. For example, cytotoxic drugs have typically been conjugated to antibodies through surface-exposed lysines or cysteines obtained by reducing interchain disulfide bonds, producing different species of ADCs with different attachment sites on the antibody and different drug to antibody ratios (DARs). The non-specific conjugation present challenges in conjugation process control, product characterization, and quality control of the ADC products. Also, the different ADC species can have very different safety and efficacy profiles. As the disulfide bonds between a heavy chain and its paired light chain of IgG1 antibodies may be the preferred bond to be reduced in the conjugation process, the resulting ADC species with light chain drug conjugates may be less stable and tend to loss their light chains in circulation.

Recently, some site-specific ADC technologies have been developed to obtain more homogeneous ADCs. For example, certain amino acids such as cysteine, unnatural amino acid residues, such as p-acetylphenylalanine (pAcF), short peptide tags which can be recognized and modified by enzymes, were engineered into select sites in the amino acid sequences of antibodies. Others tried glycan-mediated conjugation using the glycans attached on the heavy chains of the antibodies.

### SUMMARY OF THE INVENTION

In one aspect of the invention, an isolated IgG antibody is provided, which comprises: two identical heavy chains each comprising (a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue; and (b) a CH1 domain located upstream of and connected to the hinge region, the CH1 domain comprising a cysteine at the position of 142 according to the IMGT numbering scheme. The antibody can further comprise two identical kappa light chains, each paired with a heavy chain.

The CH1 domain of the IgG antibody can have the same sequence as that of the CH1 domain of a native human IgG2, IgG3, or IgG4 subclass antibody. Alternatively, the CH1 domain of the IgG antibody can have the sequence of that of the CH1 domain of a native human IgG1 antibody with the mutation S142C.

Each of the heavy chains of the IgG antibody can further comprise CH2-CH3 domains of a native human IgG1, IgG2, IgG3, IgG4 subclass antibody downstream of and connected to the hinge region. The Cl-I2-CH3 domains can optionally include one or more mutations.

In some embodiments, the amino acid sequence of the hinge is selected from the group consisting of: (a) CKTHTCPPCP (SEQ ID NO: 1); (b) DKTCHTCPPCP (SEQ ID NO: 2);
(c) ERKSCVECPPCP (SEQ ID NO: 3); (d) EPKSCDKTHTCPPCP (SEQ ID NO: 4); (e) EPKSDCKTHTCPPCP (SEQ ID NO: 5); (f) EPKSDKCTHTCPPCP (SEQ ID NO: 6); (g) EPKSDCKTHTVECPPCP (SEQ ID NO: 7); (h) EPKSDCKTVECPPCP (SEQ ID NO: 8); (i) EPKSDKCTHTVECPPCP (SEQ ID NO: 9); (j) EPPKSCDKTHTVECPPCP (SEQ ID NO: 10);
(k) EPPKSDCKTHTVECPPCP (SEQ ID NO: 11); (l) EPPPKSCDKTHTVECPPCP (SEQ ID NO: 12); and (m) EPPPPKSCDKTHTVECPPCP (SEQ ID NO: 13); and (n) EPPKSDCKTKTVECPPCP (SEQ ID NO: 28).

In some embodiments, the antibody further comprises a Fab domain that specifically binds to an antigen selected from the group consisting of EGFR, HER2, HER3, BCMA, B7H3, CEA, CEACAM6, claudin 18.2, c-MET, folate receptor, CD3, CD19, CD20, CD22, CD25, CD27L, CD30, CD33, CD37, CD48, CD56, CD70, CD73, CD74, CD79b, CD98, CD138, CD309 (VEGFR2), collagen IV, endothelin receptor ETB, ENPP3, fibronectin extra-domain B, GCC, GPNMB, LIV-1 (ZIP6), MUC1, MUC16, Mesothelin, NaPi2b, nectin 4, p-Cadherin, periostin, PSMA, SC-16 (anti-Fyn3), SLC44A4, SLTRK6, STEAP1, tenascin c, tissue factor, Trop2, and 5T4 (TPBG).

In some embodiments, the antibody has one of the following structures:
(a) two identical heavy chains each comprising an amino acid sequence of SEQ ID NO: 14; and two light chains each comprising an amino acid sequence of SEQ ID NO: 23;
(b) two identical heavy chains each comprising an amino acid sequence of SEQ ID NO: 15; and two light chains each comprising an amino acid sequence of SEQ ID NO: 23;
(c) two identical heavy chains each comprising an amino acid sequence of SEQ ID NO: 16; and two light chains each comprising an amino acid sequence of SEQ ID NO: 23;
(d) two identical heavy chains each comprising an amino acid sequence of SEQ ID NO: 17; and two light chains each comprising an amino acid sequence of SEQ ID NO: 23;
(e) two identical heavy chains each comprising an amino acid sequence of SEQ ID NO: 18; and two light chains each comprising an amino acid sequence of SEQ ID NO: 24;
(f) two identical heavy chains each comprising an amino acid sequence of SEQ NO: 19; and two light chains each comprising an amino acid sequence of SEQ ID NO: 25;
(g) two identical heavy chains each comprising an amino acid sequence of SEQ ID NO: 20; and two light chains each comprising an amino acid sequence of SEQ ID NO: 26;
(h) two identical heavy chains each comprising an amino acid sequence of SEQ ID NO: 21; and two light chains each comprising an amino acid sequence of SEQ ID NO: 25;
(i) two identical heavy chains each comprising an amino acid sequence of SEQ ID NO: 22; and two light chains each comprising an amino acid sequence of SEQ ID NO: 27; and
(j) two identical heavy chains each comprising an amino acid sequence of SEQ ID NO: 29; and two light chains each comprising an amino acid sequence of SEQ ID NO: 31; and
(k) two identical heavy chains each comprising an amino acid sequence of SEQ ID NO: 30; and two light chains each comprising an amino acid sequence of SEQ ID NO: 32.

In a further aspect, the present disclosure provides an antibody-drug conjugate (ADC) or a pharmaceutically acceptable salt thereof, comprising an antibody described herein conjugated to a cytotoxic drug by a chemical linker. In some embodiments, the cytotoxic drug is selected from the group consisting of monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, eribulin (CAS), ¹³¹I, interleukins, tumor necrosis factors, chemokines, irinotecan (SN38), exatecan, exatecan derivative, and nanoparticles.

The chemical linker linking the antibody portion and the cytotoxic drug can be cleavable or non-cleavable. In some embodiments, the linker comprises a PEGn spacer where n is between 1 and 20 (i.e., having 1 to 20 repeat units (CH₂CH₂O)). In some embodiments, the chemical linker further comprises a linker segment connected to the PEGn spacer. In some embodiments, the chemical linker comprises a linker segment but does not comprise a PEGn spacer. In some embodiments, the linker segment can be selected from the group consisting of 6-maleimidocaproyl (MC), maleimidopropionyl (MP), valine-citrulline (Val-Cit), alanine-phenylalanine (Ala-Phe), p-aminobenzyloxycarbonyl (PAB), 6-maleimidocaproyl-valine-citrulline-p-aminobenzyloxycarbonyl (MC-Val-Cit-PAB), Mal-PEGₙ-Val-Cit-PAB (n=1-20), Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate, or combinations thereof.

In certain embodiments of the ADC, each of the two heavy chains of the antibody comprises the amino acid sequence of SEQ ID NO: 21, each of the two light chains of the antibody comprises an amino acid sequence of SEQ ID NO: 25, and the cytotoxic drug is eribulin or MMAE.

In certain embodiments of the ADC, each of the two heavy chains of the antibody comprises the amino acid sequence of SEQ ID NO: 19, each of the two light chains of the antibody comprises an amino acid sequence of SEQ ID NO: 25, and the cytotoxic drug is eribulin or MMAE.

In certain embodiments of the ADC, each of the two heavy chains of the antibody comprises the amino acid sequence of SEQ ID NO: 20, each of the two light chains of the antibody comprises an amino acid sequence of SEQ ID NO: 26, and the cytotoxic drug is eribulin or MMAE.

In certain embodiments of the ADC, each of the two heavy chains of the antibody comprises the amino acid sequence of SEQ ID NO: 29, each of the two light chains of the antibody comprises an amino acid sequence of SEQ ID NO: 31, and the cytotoxic drug is eribulin or MMAE.

In certain embodiments of the ADC, each of the two heavy chains of the antibody comprises the amino acid sequence of SEQ ID NO: 30, each of the two light chains of the antibody comprises an amino acid sequence of SEQ ID NO: 32, and the cytotoxic drug is eribulin or MMAE.

In a preparation of the ADC or a pharmaceutically acceptable salt thereof, at least 80% of the ADC are conjugates between the chemical linker with the antibody through the cysteines on the heavy chains of the antibody. In some embodiments of the preparation, ADC molecules having drug to antibody ratio (DAR) of 2 accounts for more than 60% of the total amount of ADC molecules.

In a further aspect, the present disclosure provides an antibody-drug conjugate (ADC) or a pharmaceutically acceptable salt thereof, comprising the reaction product of: an antibody as described herein which has undergone at least a partial reduction such that at least some H—H disulfide bonds between the corresponding cysteines in the hinge region of the antibody are reduced to free sulfhydryls; and a chemical linker comprising a terminal thiol reactive group, attached to a cytotoxic drug molecule.

In a further aspect, the present disclosure provides a method of producing an antibody-drug conjugate (ADC) or a pharmaceutically acceptable salt thereof, comprising: at least partially reducing H—H disulfide bonds between the corresponding cysteines in the hinge region of the IgG antibody as described herein to obtain free sulfhydryls; and reacting a chemical linker containing a terminal thiol reacting group with the sulfhydryls of the cysteines in the hinge region, to thereby conjugate one or more cytotoxic drug molecules to the antibody through the chemical linker.

### BRIEF DESCRIPTION OF THE DRAWINGS

FIG. 1 shows schematic diagrams of the amino acid numbering system of antibodies as used in this application. (a) IMGT-based amino acid numbering scheme of human IgG1 (κ). (b) the numbering scheme for mutants (for the IgG1 hinge region).

FIG. 2A shows a schematic structure of an engineered antibody according to embodiments of the present invention. FIG. 2B shows schematically disulfide bonds in an IgG antibody according to embodiments of the present invention.

FIG. 3 shows characterization of example antibodies of the present invention. (a) purity and yields of antibodies produced in HEK293 cells; (b) SDS-PAGE analysis of reducing (R) and Non-Reducing (NR) antibodies; (c) SEC-HPLC analysis of purified antibodies.

FIG. 4 shows HIC profile of ADCs made of native human IgG1 (κ), IgG2 (κ) and engineered antibody examples of the present invention, a: IgG1(κ)-MMAE; b: IgG2(κ)-MMAE; c: BB0500-2a-MMAE; d: BB0500-2b-MMAE; e: BB0500-2g-MMAE; f: BB0500-2n-MMAE; g: BR0301-MMAE; h: BR0302-MMAE.

FIG. 5 shows CE-SDS profiles of certain examples of ADCs of the present invention: a: IgG1 (κ)-MMAE; b: IgG2 (κ)-MMAE; c: BB0500-2a-MMAE; d: BB0500-2g-MMAE.

FIG. 6 shows binding curves of different engineered antibodies according to embodiments of the present invention and control (wt) antibodies to EGFR (a), HER3 (b), and HER2 (c) proteins.

FIG. 7 shows cytotoxicity curves of engineered ADCs according to embodiments of the present invention to EGFR-expressing cancer cells (a: A431 cell, b: NUGC3 cell); and other engineered ADCs according to embodiments of the present invention to HER2-expressing cancer cells (c: NCI-N87 (HER2-high), and d: A431 (HER2-low)).

### DETAILED DESCRIPTION

In one aspect, the present disclosure provides a novel format of engineered antibody where its heavy chain contains a CH1 domain with a cysteine residue at or near 142th amino acid of the CH1 domain, and its hinge region comprises a sequence including three cysteines arranged in a pattern, which can be viewed as a sequence including a third or additional cysteine residue positioned upstream of the two indigenous cysteines in a human IgG1 hinge region. This third cysteine residue is herein referred to as the “HG3 cysteine.”

Antibodies containing heavy chain in this format preferably form H-L, interchain disulfide bond between C142 (or a cysteine near 142th position, according to the IMGT numbering system as further described below) of the CH1 domain with the last cysteine residue in the light chain. The cysteine residue in the hinge region upstream of the native CPPCP sequence forms a third H—H inter-chain disulfide bond. The cysteine at or near amino acid 142 in the CH1 domain could be introduced by mutation or insertion of a single amino acid in IgG1 subtype, or could come from the natural cysteine residue in the CH1 domain of IgG2, IgG3, or IgG4 subtypes. Compared with native H-L disulfide bonds in IgG1, which are between a cysteine in the hinge region of the IgG1 antibody heavy chain and the terminal end of the paired light chain, the H-L disulfide bond in this format is more stable and can be kept intact in the reducing condition during the drug conjugation to the antibody. This dramatically reduces the chances of obtaining ADCs which contains light chain drug conjugates.

The exact position of the third cysteine in the hinge region and its surrounding amino acid sequences could vary. The third H—H inter-chain disulfide bond is more prone to reduction than the indigenous H—H inter-chain disulfide bonds or the H-L inter-chain disulfide bonds. The reduction-prone property of this disulfide bond can facilitate site-specific drug conjugation. Selected reduction and drug conjugation conditions can be used to obtain predominantly site-specific drug conjugation to the third cysteine, the “HG3 cysteine”.

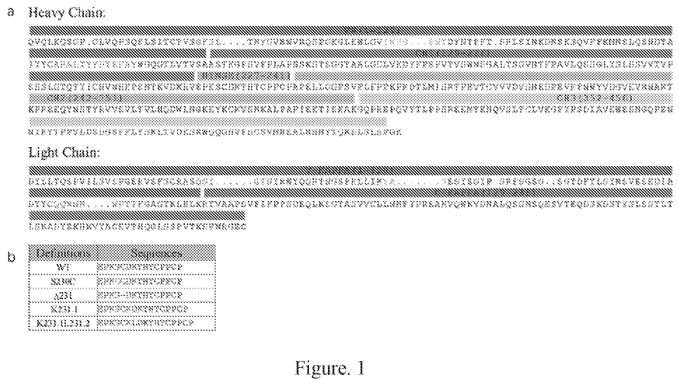

The IMGT numbering system for immunoglobulin superfamily is used herein to simplify the numbering scheme, where the VH or VL domain each contain amino acid residues 1-128. Accordingly, amino acids in the CH1 domain are numbered as aa129-226; kappa domain as aa129-235; hinge region as aa227-241 (according to IgG1); CH2 as aa242-351, and CH3 as aa352-456 (see FIG. 1a). Based on this numbering scheme, the H-L inter-chain disulfide bond in wild-type IgG1 (κ) would be formed between H (C231)-L(C235), while in IgG2(κ) (or IgG3(κ) or IgG4(κ)) it could be formed between H(C142)-L(C235). IgG1 mutant with heavy chain serein 230 changed to cysteine would be named IgG1 (S230C), while with deletion of C231 would be named IgG1 (Δ231). Insertion of a lysine after C231 would be named K231.1 and insertion of two amino acids, KL, after C231 would be named K231.1L231.2 (see FIG. 1b which shows a few examples of notations for mutations introduced in the hinge region of the IgG1).

The term “isolated antibody” as used herein refers to an antibody that is substantially free of other antibodies having different antigenic specificities. An isolated antibody that specifically binds to an antigen is substantially free of antibodies that do not bind to that antigen.

The term “monoclonal antibody” as used herein refer to a preparation of a population of antibody molecules of substantially homogeneous molecular composition, wherein the individual antibodies in the population of the antibody molecules are identical except for possible naturally occurring mutations that may be present in miniscule amounts.

In some embodiments, the present disclosure provides an isolated IgG antibody, which two identical heavy chains each comprising (a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue; and (b) a CH1 domain located upstream of and connected to the hinge region, the CH1 domain comprising a cysteine at the position of 142 according to the IMGT numbering scheme.

The IgG antibody can further comprise two identical kappa light chains. The cysteine residue at the C-terminal of the kappa light chains can form disulfide bond with the CH1 C142.

In some embodiments of the IgG antibody, the CH1 domain of the IgG antibody has the same sequence as that of the CH1 domain of a native human IgG2, IgG3, or IgG4 subclass antibody.

In some embodiments of the IgG antibody, the CH1 domain of the IgG antibody has the sequence of that of the CH1 domain of a native human IgG1 antibody with the mutation S142C.

In some embodiments of the IgG antibody, each of the heavy chains can further comprise an amino acid sequence of CH2-CH3 domains of a native human IgG1, IgG2, IgG3, IgG4 subclass antibody downstream of and connected to the hinge region.

In some embodiments, the amino acid sequence of the hinge is selected from the group consisting of: (a) CKTHTCPPCP (SEQ ID NO: 1); (b) DKTCHTCPPCP (SEQ ID NO: 2); (c) ERKSCVECPPCP (SEQ ID NO: 3); (d) EPKSCDKTHTCPPCP (SEQ ID NO: 4); (e) EPKSDCKTHTCPPCP (SEQ ID NO: 5); (f) EPKSDKCTHTCPPCP (SEQ ID NO: 6); (g) EPKSDCKTHTVECPPCP (SEQ ID NO: 7); (h) EPKSDCKTVECPPCP (SEQ ID NO: 8); (i) EPKSDKCTHTVECPPCP (SEQ ID NO: 9); (j) EPPKSCDKTHTVECPPCP (SEQ ID NO: 10); (k) EPPKSDCKTHTVECPPCP (SEQ ID NO: 11); (l) EPPPKSCDKTHTVECPPCP (SEQ ID NO: 12); (m) EPPPPKSCDKTHTVECPPCP (SEQ ID NO: 13); and (n) EPPKSDCKTKTVECPPCP (SEQ ID NO: 28)

The antibody can comprise a Fab domain that specifically binds to an antigen selected from the group consisting of EGFR, HER2, HER3, BCMA, B7H3, CEA, CEACAM6, claudin 18.2, c-MET, folate receptor, CD3, CD19, CD20, CD22, CD25, CD27L, CD30, CD33, CD37, CD48, CD56, CD70, CD73, CD74, CD79b, CD98, CD138, CD309 ('VEGFR2), collagen IV, endothelin receptor ETB, ENPP3, fibronectin extra-domain B, GCC, GPNMB, LIV-1 (ZIP6), MUC1, MUC16, Mesothelin, NaPi2b, nectin 4, p-Cadherin, periostin, PSMA, SC-16 (anti-Fyn3), SLC44A4, SLTRK6, STEAP1, tenascin c, tissue factor, Trop2, and 5T4 (TPBG).

Examples of the sequences of the IgG antibody include:
(a) two identical heavy chains each comprising an amino acid sequence of SEQ ID NO: 14; and two light chains each comprising an amino acid sequence of SEQ ID NO: 23;
(b) two identical heavy chains each comprising an amino acid sequence of SEQ ID NO: 15; and two light chains each comprising an amino acid sequence of SEQ ID NO: 23;
(c) two identical heavy chains each comprising an amino acid sequence of SEQ ID NO: 16; and two light chains each comprising an amino acid sequence of SEQ ID NO: 23;
(d) two identical heavy chains each comprising an amino acid sequence of SEQ ID NO: 17; and two light chains each comprising an amino acid sequence of SEQ ID NO: 23;
(e) two identical heavy chains each comprising an amino acid sequence of SEQ ID NO: 18; and two light chains each comprising an amino acid sequence of SEQ ID NO: 24;
(f) two identical heavy chains each comprising an amino acid sequence of SEQ ID NO: 19; and two light chains each comprising an amino acid sequence of SEQ ID NO: 25;
(g) two identical heavy chains each comprising an amino acid sequence of SEQ ID NO: 20; and two light chains each comprising an amino acid sequence of SEQ ID NO: 26;
(h) two identical heavy chains each comprising an amino acid sequence of SEQ ID NO: 21; and two light chains each comprising an amino acid sequence of SEQ TD NO: 25;
(i) two identical heavy chains each comprising an amino acid sequence of SEQ ID NO: 22, and two light chains each comprising an amino acid sequence of SEQ ID NO: 27;
(j) two identical heavy chains each comprising an amino acid sequence of SEQ ID NO: 29; and two light chains each comprising an amino acid sequence of SEQ ID NO: 31; and
(k) two identical heavy chains each comprising an amino acid sequence of SEQ ID NO: 30; and two light chains each comprising an amino acid sequence of SEQ ID NO: 32.

DNA encoding an amino acid sequence variant of a starting polypeptide can prepared by a variety of methods known in the art. These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may be constructed also by restriction fragment manipulation or by overlap extension PCR with synthetic oligonucleotides. Mutagenic primers encode the cysteine codon replacement(s). Standard mutagenesis techniques can be employed to generate DNA encoding such mutant engineered antibodies.

In yet a further aspect, the present disclosure provides a nucleic acid molecule encoding the antibody or antigen-binding portion thereof of any of the antibody described herein. A host cell (e.g., a CHO cell, a lymphocytic cell, a human embryonic kidney cell, or microorganisms, such as E. coli, and fungi, such as yeast) containing an expression vector containing the nucleic acid molecule, can be used to produce antibodies of the present disclosure, preferably monoclonal antibodies. In one embodiment, DNA encoding partial or full-length antibody of the present disclosure can be obtained by standard molecular biology techniques is inserted into one or more expression vectors such that the genes are operatively linked to transcriptional and translational regulatory sequences. The term “operatively linked” is intended to mean that an antibody gene is ligated into a vector such that transcriptional and translational control sequences within the vector serve their intended function of regulating the transcription and translation of the antibody gene. The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody encoding DNA can be inserted into the expression vector. The recombinant expression vector can encode a signal peptide that facilitates secretion of the antibody chain from a host cell. The antibody encoding DNA can be cloned into the vector such that the signal peptide is linked in-frame to the amino terminus of the antibody encoding DNA. The signal peptide can be an immunoglobulin signal peptide or a heterologous signal peptide (i.e., a signal peptide from a non-immunoglobulin protein).

In a further aspect, an antibody-drug conjugate (ADC) or a pharmaceutically acceptable salt thereof, is provided, which comprises an antibody of the present disclosure as described herein, conjugated to a cytotoxic drug by a chemical linker. The cytotoxic drug can be selected from the group consisting of monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, eribulin, ¹³¹I, interleukins, tumor necrosis factors, chemokines, irinotecan (SN38), exatecan, exatecan derivative, and nanoparticles.

The chemical linker linking the antibody portion and the cytotoxic drug can be cleavable or non-cleavable. In some embodiments, the linker comprises a PEGn spacer where n is between 1 and 20 (i.e., having 1 to 20 repeat units (CH₂CH₂O)). In some embodiments, the chemical linker further comprises a linker segment connected to the PEGn spacer. In some embodiments, the chemical linker comprises a linker segment but does not comprise a PEGn spacer. In some embodiments, the chemical linker can include a segment that is selected from the group consisting of 6-maleimidocaproyl (MC), maleimidopropionyl (MP), valine-citrulline (Val-Cit), alanine-phenylalanine (Ala-Phe), p-aminobenzyloxycarbonyl (PAB), 6-maleimidocaproyl-valine-citrulline-p-aminobenzyloxycarbonyl (MC-Val-Cit-PAB), Mal-PEGₙ-Val-Cit-PAB (n=1-20), Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate, or combinations thereof.

In certain embodiments of the ADC, each of the two heavy chains of the antibody comprises the amino acid sequence of SEQ ID NO: 21, each of the two light chains of the antibody comprises an amino acid sequence of SEQ ID NO: 25, and the cytotoxic drug is eribulin or MMAE.

In certain embodiments of the ADC, each of the two heavy chains of the antibody comprises the amino acid sequence of SEQ ID NO: 19, each of the two light chains of the antibody comprises an amino acid sequence of SEQ ID NO: 25, and the cytotoxic drug is eribulin or MMAE.

In certain embodiments of the ADC, each of the two heavy chains of the antibody comprises the amino acid sequence of SEQ ID NO: 20, each of the two light chains of the antibody comprises an amino acid sequence of SEQ ID NO: 26, and the cytotoxic drug is eribulin or MMAE.

In certain embodiments of the ADC, each of the two heavy chains of the antibody comprises the amino acid sequence of SEQ ID NO: 29, each of the two light chains of the antibody comprises an amino acid sequence of SEQ ID NO: 31, and the cytotoxic drug is eribulin or MMAE.

In certain embodiments of the ADC, each of the two heavy chains of the antibody comprises the amino acid sequence of SEQ ID NO: 30, each of the two light chains of the antibody comprises an amino acid sequence of SEQ ID NO: 32, and the cytotoxic drug is eribulin or MMAE.

In the present disclosure, the pharmaceutically acceptable salts of the ADCs include acid addition salts of inorganic acids, carboxylic acids and sulfonic acids, for example, salts of the following acids: hydrochloric acid, hydrobromic acid, sulfuric acid, phosphoric acid, methanesulfonic acid, ethanesulfonic acid, benzenesulfonic acid, toluenesulfonic acid, naphthalene disulfonic acid, acetic acid, trifluoroacetic acid, propionic acid, lactic acid, tartaric acid, malic acid, citric acid, fumaric acid, maleic acid and benzoic acid.

The pharmaceutically acceptable salts of the antibody-drug conjugates of the present disclosure also include salts of conventional bases, for example alkali metal salts (e.g., sodium salts and potassium salts), alkaline earth metal salts (e.g., calcium salts and magnesium salts) and ammonium salts derived from ammonia or organic amines containing from 1 to 16 carbon atoms, in which the organic amines are, for example, ethylamine, diethylamine, triethylamine, ethyl diisopropylamine, monoethanolamine, diethanolamine, triethanolamine, dicyclohexylamine, dimethylaminoethanol, procaine, dibenzamide, N-methylpiperidine, N-methylmorpholine, arginine, lysine and 1,2-ethylenediamine.

It is understood that an ADC as used herein refers to a molecule that contains both a drug molecule and an antibody (or an antigen binding portion thereof) where the drug and the antibody (or the antigen binding portion thereof) is covalently connected by a linker. An “ADC preparation” herein refers to a collection or population of ADC molecules whose structure may differ due to possibly different attachment sites of the chemical linker to the antibody (or the antigen binding portion thereof). In some embodiments, the chemical linker is primarily or predominantly (e.g., ≥80%, ≥85%, ≥90%, ≥95%, or ≥98%) conjugated with cysteines on a heavy chain, resulting in an ADC preparation that is substantially devoid of light chain conjugation. In some embodiments, the chemical linker is conjugated with the antibody predominantly through the cysteines in the hinge region of the heavy chains of the antibody. And in certain embodiments, ADC molecules having drug to antibody ratio (DAR) of 2 accounts for at least 60%, at least 70%, at least 80%, at least 85%, or at least 90% of the total amount of ADC molecules.

In a further aspect, the ADC or pharmaceutically acceptable salt thereof is or comprises the reaction product of an antibody of the present invention as described herein, which has undergone at least a partial reduction such that at least some H—H disulfide bonds between the corresponding cysteines in the hinge region of the antibody are reduced to free sulfhydryls; and a chemical linker comprising a terminal thiol reactive group attached to a cytotoxic drug molecule.

In a further aspect, a method of producing the ADC or pharmaceutically acceptable salt thereof is provided, in which the H—H disulfide bonds between the corresponding cysteines in the hinge region of an antibody of the present invention is at least partially reduced to obtain free sulfhydryls; and reacting a chemical linker containing a terminal thiol reactive group with the sulfhydryls of the cysteines in the hinge region, to thereby conjugate one or more cytotoxic drug molecules to the antibody through the chemical linker.

In further aspect, the present disclosure provides a pharmaceutical composition comprising one or more antibodies, ADCs or the pharmaceutically acceptable salts thereof, of the present invention, together with a pharmaceutically acceptable carrier. As used herein, “pharmaceutically acceptable carrier” includes pharmaceutically acceptable carriers, excipients or stabilizers. These include but are not limited solvents, dispersion media, coatings, antibacterial and antifungal agents, isotonic and absorption delaying agents, surface active agents, thickening or emulsifying agents, solid binders, dispersion or suspension aids, solubilizers, colorants, flavoring agents, coatings, disintegrating agents, lubricants, sweeteners, preservatives, isotonic agents, and the like that are physiologically compatible. The selection of suitable carrier is within the knowledge of an artisan skilled in the art.

The composition may comprise one or more additional pharmaceutically active ingredients, such as another antibody, a drug, e.g., a cytotoxic or anti-tumor agent. The pharmaceutical compositions of the invention also can be administered in a combination therapy with, for example, another anti-cancer agent, another anti-inflammatory agent, etc.

The pharmaceutical composition can be suitable for intravenous, intramuscular, subcutaneous, parenteral, epidermal, and other routes of administration. Depending on the route of administration, the active ingredient can be coated with a material or otherwise loaded in a material or structure to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the composition of the invention can be administered via a non-parenteral route, such as a topical, epidermal or mucosal route of administration, e.g., intranasally, orally, vaginally, rectally, sublingually or topically.

### EXAMPLES

### 1. Design of the Engineered Antibodies

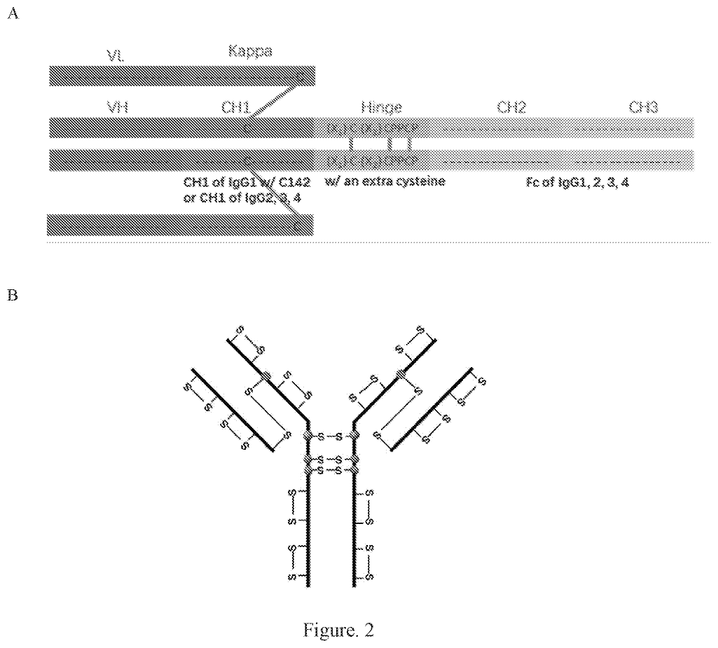

Antibodies in Table 1 were designed and prepared, where CH1 domain of these antibodies contained a cysteine residue at amino acid 142. In some antibodies, the cysteine at amino acid 142 in CH1 domain was introduced by mutation or insertion of a single amino acid (S142C in Table 1 cysteine substitution for 142 serine), in other antibodies the C142 came from the natural cysteine residue in CH1 domain of IgG2 subtypes (denoted G2CH1 in Table 1). In place of the natural hinge region, these antibodies each include an artificial hinge sequence with a third cysteine residue (HG3 cysteine) located upstream of the CPPCP sequence. The amino acid sequences surrounding the HG3 cysteine residue can vary among different engineered antibodies. With a cysteine residue introduced at position 142 in the CH1 of the engineered antibodies, the H-L interchain disulfide bond can be formed between C142 of the CH1 domain with C235 in a light chain. The HG3 cysteine can pair with the cysteine at the same position in the other H chain and formed an extra H—H inter-chain disulfide bond in addition to the indigenous H—H inter-chain disulfide bonds (FIG. 2).

TABLE 1
List of Engineered Antibodies

| Clone ID | Target | C142 source | Hinge Sequences | Hinge SEQ ID No. |
|---|---|---|---|---|
| BB0500-1 | EGFR | G2CH1 | CKTHTCPPCP | 1 |
| BB0500-2 | EGFR | G2CH1 | DKTCHTCPPCP | 2 |
| BB0500-6 | EGFR | G2CH1 | DKTCHTCPPCP | 2 |
| BB0500-3 | EGFR | G2CH1 | ERKSCVECPPCP | 3 |
| BB0500-5 | EGFR | G1(S142C) | EPKSCDKTHTCPPCP | 4 |
| BB0500-2a | EGFR | G2CH1 | EPKSCDKTHTCPPCP | 4 |
| BB0500-7 | EGFR | G2CH1 | EPKSCDKTHTCPPCP | 4 |
| BB0500-2b | EGFR | G2CH1 | EPKSDCKTHTCPPCP | 5 |
| BB0500-7a | EGFR | G2CH1 | EPKSDCKTHTCPPCP | 5 |
| BB0500-2c | EGFR | G2CH1 | EPKSDKCTHTCPPCP | 6 |
| BB0500-6a | EGFR | G2CH1 | EPKSDKCTHTCPPCP | 6 |
| BB0500-7b | EGFR | G2CH1 | EPKSDKCTHTCPPCP | 6 |
| BB0500-2d | EGFR | G2CH1 | EPKSDCKTHTVECPPCP | 7 |
| BB0500-2e | EGFR | G2CH1 | EPKSDCKTVECPPCP | 8 |
| BB0500-2f | EGFR | G2CH1 | EPKSDKCTHTVECPPCP | 9 |
| BB0500-2g | EGFR | G2CH1 | EPPKSCDKTHTVECPPCP | 10 |
| BB0801 | HER3 | G1(S142C) | EPPKSCDKTHTVECPPCP | 10 |
| BB0500-2n | EGFR | G2CH1 | EPPKSDCKTHTVECPPCP | 11 |
| BB0802 | HER3 | G1(S142C) | EPPPKSCDKTHTVECPPCP | 12 |
| BB0803 | HER3 | G1(S142C) | EPPPPKSCDKTHTVECPPCP | 13 |
| BR0301 | HER2 | G2CH1 | EPPKSDCKTKTVECPPCP | 28 |
| BR0302 | HER2 | G2CH1 | EPPKSDCKTKTVECPPCP | 28 |

Structure and sequences of example engineered antibodies are described as follows:
1. BB0500-3 is made in format of IgG2κ isotype, where its heavy chain contains: VII (of anti-EGFR)-CH1 (of IgG2)-hinge (SEQ ID NO: 3)-CH2CH3 (of IgG2), and light chain contains: VL-CL (κ). Sequence of the heavy chain of BB0500-3 is shown as SEQ ID NO: 14. Sequence of the light chain of BB0500-3 is shown as SEQ ID NO: 23.
2. BB0500-5 is made in format of IgG_{1κ} isotype, where its heavy chain contains: VET (of anti-EGFR)-CH1 (S142C)-hinge (SEQ ID NO: 4)-CH2CH3 (of IgG1), and light chain contains: VL-CL (κ). Sequence of the heavy chain of BB0500-5 is shown as SEQ ID NO: 15. Sequence of the light chain of BB0500-5 is shown as SEQ ID NO: 23.
3. BB0500-2a is made in format of IgG_{1κ} isotype, where its heavy chain contains: VH (of anti-EGFR)-CH1 (of IgG2)-hinge (SEQ ID NO: 4)-CH2CH3 (of IgG1), and light chain contains: VL-CL (κ). Sequence of the heavy chain of BB0500-2a is shown as SEQ ID NO: 16. Sequence of the light chain of BB0500-2a is shown as SEQ ID NO: 23.
4. BB0500-2b is made in format of IgG_{1κ} isotype, where its heavy chain contains: VH (of anti-EGFR)-CH1 (of IgG2)-hinge (SEQ ID NO: 5)-CH2CH3 (of IgG1), and light chain contains: VL-CL (κ). Sequence of the heavy chain of BB0500-2b is shown as SEQ ID NO: 17. Sequence of the light chain of BB0500-2b is shown as SEQ ID NO: 23.
BB0500-6a is made in format of IgG_{1κ} isotype, where its heavy chain contains: VH (of anti-EGFR)-CH1 (of IgG2)-hinge (SEQ ID NO: 6)-CH2CH3 (of IgG1), and light chain contains: VL-CL (κ). Sequence of the heavy chain of BB0500-6a is shown as SEQ ID NO: 18. Sequence of the light chain of BB0500-6a is shown as SEQ ID NO: 24.
6. BB0500-2f is made in format of IgG_{2κ} isotype, where its heavy chain contains: VH (of anti-EGFR)-CH1 (of IgG2)-hinge (SEQ ID NO: 9)-CH2CH3 (of IgG2), and light chain contains: VL-CL (κ). Sequence of the heavy chain of BB0500-2f is shown as SEQ ID NO: 19. Sequence of the light chain of BB0500-2f is shown as SEQ ID NO: 25.
7. BB0500-2g is made in format of IgG_{2κ} isotype, where its heavy chain contains: VH (of anti-EGFR)-CH1 (of IgG2)-hinge (SEQ ID NO: 10)-CH2CH3 (of IgG2), and light chain contains: VL-CL (κ). Sequence of the heavy chain of BB0500-2g is shown as SEQ ID NO: 20. Sequence of the light chain of BB0500-2g is shown as SEQ ID NO: 26.
8. BB0500-2n is made in format of IgG_{1κ} isotype, where its heavy chain contains: VH (of anti-EGFR)-CH1 (of IgG2)-hinge (SEQ ID NO: 11)-CH2CH3 (of IgG1), and light chain contains: VL-CL (κ). Sequence of the heavy chain of BB0500-2n is shown as SEQ ID NO: 21. Sequence of the light chain of BB0500-2n is shown as SEQ II) NO: 25.
9. BB0802 is made in format of IgG_{1κ} isotype, where its heavy chain contains: VH (of anti-HER3)-CH1 (S142C)-hinge (SEQ ID NO: 12)-CH2CH3 (of IgG1), and light chain contains: VL-CL (κ). Sequence of the heavy chain of BB0802 is shown as SEQ ID NO: 22. Sequence of the light chain of BB0802 is shown as SEQ ID NO: 27.
10. BR0301 is made in format of IgG_{1κ} isotype, where its heavy chain contains: VH (of anti-HER2)-CH1 (of IgG2)-hinge (SEQ ID NO: 28)-CH2CH3 (of IgG1), and light chain contains: VL-CL (κ). Sequence of the heavy chain of BR0301 is shown as SEQ ID NO: 29. Sequence of the light chain of BR0302 is shown as SEQ ID NO: 31.
11. BR0302 is made in format of IgG_{1κ} isotype, where its heavy chain contains: VH (of anti-HER2)-CH1 (of IgG2)-hinge (SEQ ID NO: 28)-CH2CH3 (of IgG1), and light chain contains: VL-CL (κ). Sequence of the heavy chain of BR0302 is shown as SEQ ID NO: 30. Sequence of the light chain of BR0302 is shown as SEQ ID NO: 32.

### 2. Expression and Purification of the Engineered Antibodies

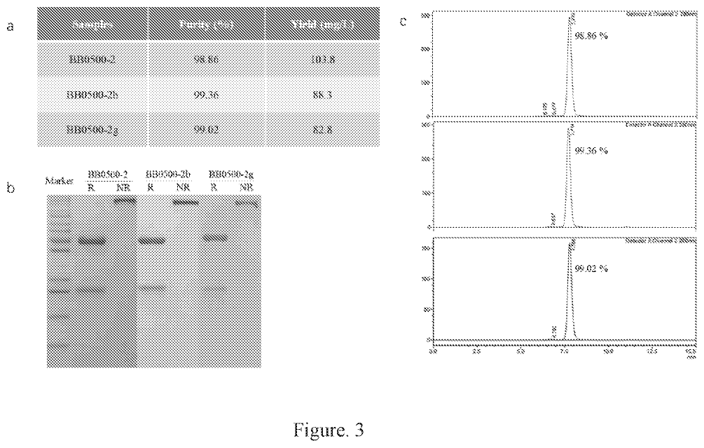

For expression of the engineered antibodies, codon optimization and gene synthesis were performed. Specific full-length heavy chain and light chain DNA were each cloned into a separate pcDNA3 plasmid. HEK293 cell transient transfection of the paired plasmids and one-step Protein A purification was used to prepare sufficient amount of proteins for testing. Antibodies made in this format expressed well with good yield and could be purified in high purity with one step protein A purification process (FIG. 3a, 3b, 3c).

### 3. Profiles of Antibody Drug Conjugates Made of Native IgG1 (κ) and IgG2 (κ)

Under certain mild reduction conditions (tris (2-chloroethyl) phosphate (TCEP): mAb=1-3, neutral pH, room temperature for <240 min), interchain disulfide bonds of IgG antibodies could be partially reduced and conjugated with a chemical linker to form ADCs. Purified antibodies were conjugated to the cytotoxic agents (e.g., MMAE, DM1, eribulin) via a linker. Antibody in phosphate buffer at neutral pH was added TCEP for partial reduction. Drug linker (MC-Val-Cit-PAB-MMAE or MC-DM1 or Mal-PEG₂-Val-Cit-PAB-eribulin) in DMA was added and allowed to react with antibody to obtain desired drug-to-antibody ratio (DAR). To characterize the antibodies and ADCs, Hydrophobic Interaction Chromatography (HIC) was performed for the evaluation of drug distribution and molar ratio of drug to antibody in ADC. CE-SDS of non-reducing ADC was also performed to evaluate percentage of non-covalently linked components in the ADC product, like free light chains (L), free heavy chains (H), half antibodies (HL), intact antibodies (LHHL) and antibodies missing one or two light chains (HHL or HH).

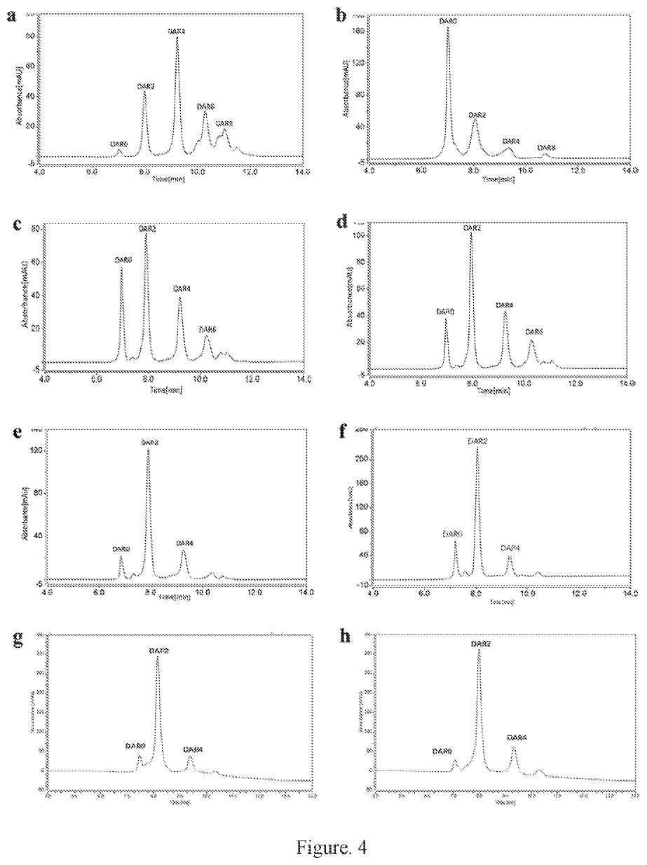

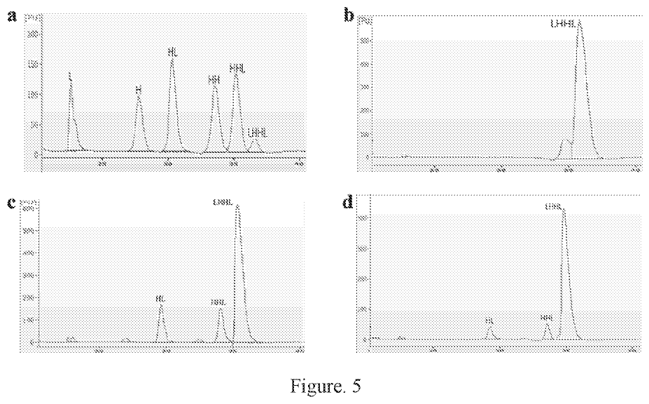

HIC profile of an IgG1 (κ) ADC revealed that there was very little naked antibody (DAR0) left at the end of the reaction period and the resulting ADC product was mixtures of DAR2-8 species (FIG. 4a). CE-SDS results of non-reduced ADC product revealed that there were high percentages of free light chains (L, 18%) and structures missing one or both light chains (20% +18%), indicating that a high percentage of conjugations occurring at the cysteine residues forming the H-L inter-chain disulfide bonds (FIG. 5a).

In contrast, under similar reduction and conjugation conditions. IgG2 (κ) is considerably more resistant to reactions. Very low percentages of either H-L or H—H inter-chain disulfide bonds were opened and majority of the IgG2 molecules remained as DAR0 at the end of the reaction period (FIG. 4b). CE-SDS profile revealed that there was high percentage of intact antibodies (LHHL, 89%), while an extremely low level of free light chains (L, ˜1%, indication of light chain drug conjugates) in the IgG2 (κ) ADC product (FIG. 5b). These results suggested that the disulfide bonds in an IgG-2 (κ) molecule are much more stable than those in IgG-1 antibodies.

### 4. Drug Conjugation to Engineered Antibodies

HIC profile revealed that ADC product based on the engineered antibodies (BB0500-2a, 2b as examples in FIG. 4c, d) were made up of mixtures of DAR0-10 species, where DAR2 specie occupied the highest percentage (35-50%), followed with DAR0 (note DAR0 refers to the antibody (or portion thereof) which was not converted to ADC in the conjugation process), DAR4, and DAR6 species (in the range of 10-25%). DAR8-10 species occupied very small percentage in the mixture (<5%) (FIG. 4c, d). CE-SDS results confirmed the overall reduction-resistant property of the engineered antibodies in comparison with IgG1 (κ), especially the H-L, inter-chain disulfide bond with only ˜2% of free light chains (indicating conjugated light chains) (FIG. 5c). Thus, the resulting ADC products were made predominantly of heavy chain only conjugates.

### 5. Site-Selective Drug Conjugation

The ADCs made of certain engineered antibodies of the present disclosure were predominantly DAR2 ADC species (about 60-70% shown in FIGS. 4e & 4f, where the antibodies in the ADCs are BB0500-2g and BB0500-2n, respectively, and about 70-80% shown in FIGS. 4g and 4h, where the antibodies in the ADCs are BR0301 and BR0302, respectively). It is believed that the sequences in the hinge region in these clones make the H—H inter chain disulfide bond between the HG3 cysteines more susceptible to reduction than the rest of the inter chain disulfide bonds, making HG3 cysteines preferred or predominant sites for drug conjugation. Antibodies containing different hinge amino acid sequences surrounding the HG3 cysteine required different reduction and conjugation conditions to achieve the DAR2-dominant ADC product. CE-SDS profile revealed that there was an extremely low level (˜1%) of free light chains (indication of light chain drug conjugates) in these ADC products (FIG. 5d).

The disulfide bond pattern of engineered antibodies was evaluated by LC-MS and LC-MS/MS analysis with trypsin digestion under non-reducing conditions. All of the disulfide bond linked peptides were identified by their molecular weights and sequences. The overall sequence coverage was 100% when combining LC-UV and LC-MS detection. The disulfide bond between light chain and heavy chain containing Cys 142, and extra disulfide bonds between two heavy chains derived from the additional Cys upstream of the CPPCP sequence in the hinge region were confirmed. Other 14 disulfide bond linkages are consistent with a typical IgG1 type disulfide bond linkage.

Upon the conjugation, the conjugation sites were determined by the characterization of the late-eluting peaks in the peptide mapping profile. The peak assignment was based on the observed masses and MS/MS spectrum of the conjugated peptides. The predominant conjugation site was determined to be the additional Cys residue upstream of the CPPCP sequence on the heavy chain in the hinge region. Low levels of conjugation at Cys residues in the CPPCP sequence in the hinge region were also detected. This is in agreement with the expected conjugation sites at inter-chain disulfide bonds between two heavy chains in the hinge region.

### 6. Binding Activity of Different Engineered Antibodies to EGFR, HER2 or HER3 Protein

ELISA assay was used to exam and compare the EGFR, HER2 or HER3 binding capabilities between the engineered antibodies and control wild type (wt) antibodies. Human EGFR, HER2 and HER3 proteins were coated onto 96-well plates and the plates were incubated overnight. Engineered antibody samples or corresponding control antibodies were diluted. Diluted samples were then transferred to proteins-coated plates and incubated at room temperature. Using HRP-labeled goat anti-human IgG Fc antibody (Sigma) as a detection agent and TMB for colorimetric reaction, the plates read at 450/650 nm for absorbance on Microplate Reader (Molecular Devices, SpectraMax 190) and data analysis was performed using a dose response curve format four parameters logistic model.

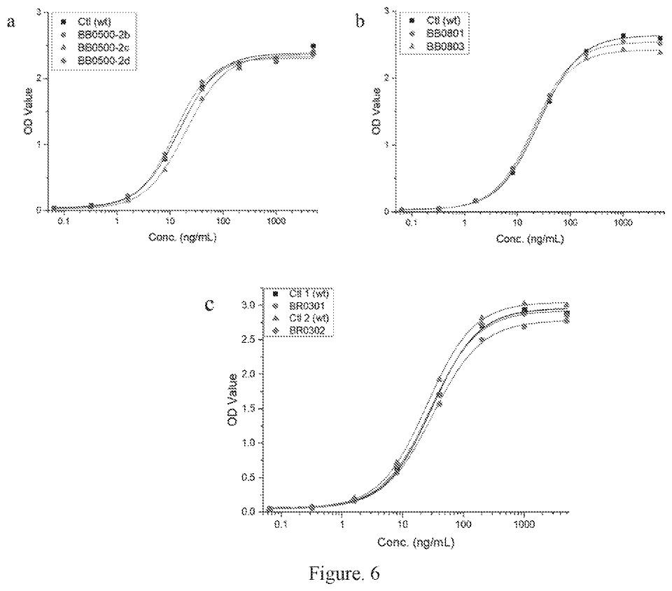

The result in FIG. 6 showed that the engineered antibodies of different targets have similar binding activity in comparison to the corresponding controls, suggesting that the novel format of engineered antibody does not affect the activity of the engineered antibodies to target proteins.

### 7. Cytotoxicity of ADC Made of Engineered Antibody to EGFR High/Low-Expressing Cells

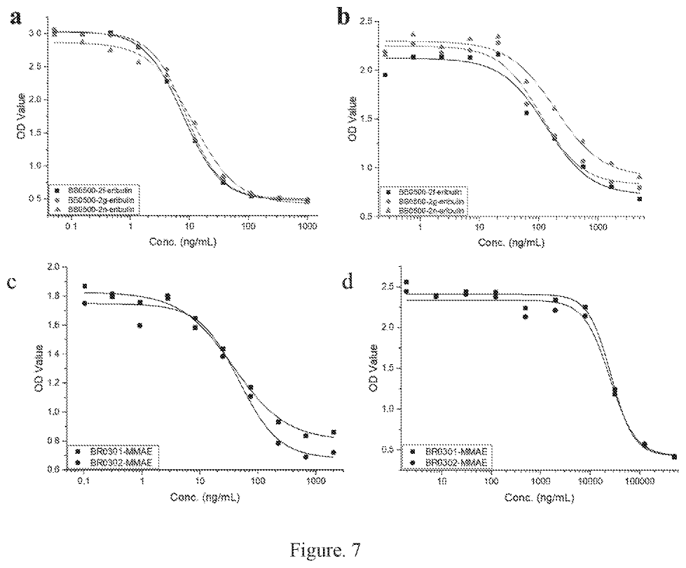

To investigate the cytotoxicity of the ADCs derived from engineered antibodies, in vitro cytotoxicity to target-expressing cancer cells was evaluated in a colorimetric-based cytotoxic assay. To perform the assay, target cells were seeded into a 96-well flat-bottom tissue culture plate at an optimized cell density for each cell line and incubated at 37° C., 5% CO₂ overnight (16-20 hrs). Serial dilutions of the ADC samples were transferred to cell plates and the assay plates were incubated for a defined period of time (3-5 days depend on cell lines) for optimal killing. Data analysis was performed using a dose response curve by four parameters logistic model. The results in FIG. 7 showed that, eribulin-containing EGFR-targeting ADC comprising made of engineered antibody portion of BB0500-2f, 2g or 2n exerted potent cytotoxicity activities to EGFR high-expressing cells (A431 cells, FIG. 7a) and EGFR low-expressing cells (NUGC3 cells, FIG. 7b). Similarly, the result for MMAE-containing HER2-targeting ADC comprising antibody portion of BB0301 or BB0302 exerted potent cytotoxicity activity to HER2 high-expressing cells (NCI-N87 cells, FIG. 7c) and HER2 low-expressing cells (A431 cells, FIG. 7d).

Sequence Listing

SEQ ID NOs: 1-13, and 28 are provided in Table 1.
SEQ ID NO: 14 (BB0500-3 heavy chain):
QVQLQESGPGLVKPSETLSLTCTVSGGSVSSGDYYWTWIRQSPGKGLEWIGHIYYSGNT
NYNPSLKSRLTISIDTSKTQFSLKLSSVTAADTAIYYCVRDRVTGAFDIWGQGTMVTVSS
ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVERKSCVECPPCPAPPVAGPSVFL
FPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTK
NQVSLTCLVKGFYPSDISVEWESNGQPENNYKTTPPMLDSDGSFFLYSKLTVDKSRWQQ
GNVFSCSVMHEALHNHYTQKSLSLSPGK
SEQ ID NO: 15 (BB0500-5 heavy chain):
QVQLQESGPGLVKPSETLSLTCTVSGGSVSSGDYYWTWIRQSPGKGLEWIGHIYYSGNT
NYNPSLKSRLTISIDTSKTQFSLKLSSVTAADTAIYYCVRDRVTGAFDIWGQGTMVTVSS
ASTKGPSVFPLAPCSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 16 (BB0500-2a heavy chain):
QVQLQESGPGLVKPSETLSLTCTVSGGSVSSGDYYWTWIRQSPGKGLEWIGHIYYSGNT
NYNPSLKSRLTISIDTSKTQFSLKLSSVTAADTAIYYCVRDRVTGAFDIWGQGTMVTVSS
ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVEPKSCDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 17 (BB0500-2b heavy chain):
QVQLQESGPGLVKPSETLSLTCTVSGGSVSSGDYYWTWIRQSPGKGLEWIGHIYYSGNT
NYNPSLKSRLTISIDTSKTQFSLKLSSVTAADTAIYYCVRDRVTGAFDIWGQGTMVTVSS
ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVEPKSDCKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 18 (BB0500-6a heavy chain):
QVQLKQSGPGLVQPSQSLSITCTVSGFSLTNYGVHWVRQSPGKGLEWLGVIWSGGNTDY
NTPFTSRLSINKDNSKSQVFFKMNSLQSDDTAIYYCARALTYYDYEFAYWGQGTLVTVS
AASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVEPKSDKCTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 19 (BB0500-2f heavy chain):
QVQLQESGPGLVKPSETLSLTCTVSGGSVSSGDYYWTWIRQSPGKGLEWIGHIYYSGNA
NYNPSLKSRLTISIDTSKTQFSLKLSSVTAADTAIYYCVRDRVTGAFDIWGQGTMVTVSS
ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVEPKSDKCTHTVECPPCPAPPVAG
PSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSRE
EMTKNQVSLTCLVKGFYPSDISVEWESNGQPENNYKTTPPMLDSDGSFFLYSKLTVDKSR
WQQGNVFSCSVMHEALHNHYTQKSLSLSPGK
SEQ ID NO: 20 (BB0500-2g heavy chain):
QVQLQESGPGLVKPSETLSLTCTVSGGSVSSGDYYWTWIRQSPGKGLEWIGHIYYSGNT
NYNPSLKSRLTISIDTSKTQFSLKLSSVTAADTAIYYCVRDRVAGAFDIWGQGTMVTVSSA
STKGPSVFPLAPCSRSTSESTAALGCLVKDYFPEPVTVSWNSGALTSGVHTFPAVLQSSGL
YSLSSVVTVPSSNFGTQTYTCNVDHKPSNTKVDKTVEPPKSCDKTHTVECPPCPAPPVAG
PSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSRE
EMTKNQVSLTCLVKGFYPSDISVEWESNGQPENNYKTTPPMLDSDGSFFLYSKLTVDKSR
WQQGNVFSCSVMHEALHNHYTQKSLSLSPGK
SEQ ID NO: 21 (BB0500-2n heavy chain):
QVQLQESGPGLVKPSETLSLTCTVSGGSVSSGDYYWTWIRQSPGKGLEWIGHIYYSGNA
NYNPSLKSRLTISIDTSKTQFSLKLSSVTAADTAIYYCVRDRVTGAFDIWGQGTMVTVSS
ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVEPPKSDCKTHTVECPPCPAPEAA
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 22 (BB0802 heavy chain):
EVQLLESGGGLVQPGGSLRLSCAASGFTFSAYGMGWVRQAPGKGLEWVSYISPSGGHT
KYADSVKGRFTISRDNSKNTLYLQMNSLRAEDTAVYYCAKVLETGLLVDAFDIWGQGT
MVTVSSASTKGPSVFPLAPCSKSTSGGTAALGCLVKDYFPEPVTVSWNSGALTSGVHTFP
AVLQSSGLYSLSSVVTVPSSSLGTQTYICNVNHKPSNTKVDKRVEPPPKSCDKTHTVECPP
CPAPELLGGPSVFLFPPKPKDTLMISRTPEVTCVVVDVSHEDPEVKENWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 23 (light chain):
DIQMTQSPSSLSASVGDRVTITCQASQDISNYLNWYQQKPGKAPKLLIYDASNLETGVPS
RFSGSGSGTDFTFTISSLQPEDIATYFCQHFDHLPLAFGGGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 24 (light chain):
DILLTQSPVILSVSPGERVSFSCRASQSIGTNIHWYQQRINGSPRLLIKYASESISGIPSRFSG
SGSGTDFTLSINSVESEDIADYYCQQNNNWPTTFGAGTKLELKRTVAAPSVFIFPPSDEQL
KSGTASVVCLLNNFYPREAKVQWKVDNALQSGNSQESVTEQDSKDSTYSLSSTLTLSKA
DYEKHKVYACEVTHQGLSSPVTKSFNRGEC
SEQ ID NO: 25 (light chain):
DIQMTQSPSSLSASVGDRVTITCQASQDISNYLNWYQQKPGKAPKLLIYAASNLETGVPS
RFSGSGSGTDFTFTISSLQPEDIATYFCQHFDHLPLAFGGGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 26 (light chain):
DIQMTQSPSSLSASVGDRVTITCQASQDISNALNWYQQKPGKAPKLLIYDASNLETGVPS
RFSGSGSGTDFTFTISSLQPEDIATYFCQHFDHLPLAFGGGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 27 (light chain):
QYELTQPPSVSVYPGQTASITCSGDQLGSKFVSWYQQRPGQSPVLVMYKDKRRPSEIPER
FSGSNSGNTATLTISGTQAIDEADYYCQAWDSSTYVFGTGTKVTVLRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
SEQ ID NO: 29 (BR0301 heavy chain):
EVQLVESGGGLVQPGGSLRLSCAASGFTFTDYTMDWVRQAPGKGLEWVADVNPNSGGS
IYNQRFKGRFTLSVDRSKNTLYLQMNSLRAEDTAVYYCARNLGPSFYFDYWGQGTLVT
VSSASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVEPPKSDCKTKTVECPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 30 (BR0302 heavy chain):
EVQLVESGGGLVQPGGSLRLSCAASGFNIKDTYIHWVRQAPGKGLEWVARIYPTNGYTR
YADSVKGRFTISADTSKNTAYLQMNSLRAEDTAVYYCSRWGGDGFYAMDYWGQGTLV
TVSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVEPPKSDCKTKTVECPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 31 (BR0301 light chain):
DIQMTQSPSSLSASVGDRVTITCKASQDVSIGVAWYQQKPGKAPKLLIYSASYRYTGVPS
RFSGSGSGTDFTLTISSLQPEDFATYYCQQYYTYPYTFGQGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 32 (BR0302 light chain):
DIQMTQSPSSLSASVGDRVTITCRASQDVNTAVAWYQQKPGKAPKLLIYSASFLYSGVPS
RFSGSRSGTDFTLTISSLQPEDFATYYCQQHYTTPPTFGQGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC

1. An isolated IgG antibody, comprising:
two identical heavy chains each comprising
(a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue; and
(b) a CH1 domain located upstream of and connected to the hinge region, the domain comprising a cysteine at the position of 142 according to the IMGT numbering scheme.

2. The IgG antibody of claim 1, further comprising two identical kappa light chains.

3. The IgG antibody of any of claims 1-2, wherein the CH1 domain of the IgG antibody has the same sequence as that of the CH1 domain of a native human IgG2, IgG3, or IgG4 subclass antibody.

4. The IgG antibody of any of claims 1-2, wherein the CH1 domain of the IgG antibody has the sequence of that of the CH1 domain of a native human IgG1 antibody with the mutation S142C.

5. The IgG antibody of any of claims 1-4, wherein each of the heavy chains further comprises CH2-CH3 domains of a native human IgG1, IgG2, IgG3, IgG4 subclass antibody downstream of and connected to the hinge region, wherein the CH2-CH3 optionally include one or more mutations.

6. The IgG antibody of any of claims 1-5, wherein the amino acid sequence comprised in the hinge is selected from the group consisting of:
(a)
(SEQ ID NO: 1)
CKTHTCPPCP;
(b)
(SEQ ID NO: 2)
DKTCHTCPPCP;
(c)
(SEQ ID NO: 3)
ERKSCVECPPCP;
(d)
(SEQ ID NO: 4)
EPKSCDKTHTCPPCP;
(e)
(SEQ ID NO: 5)
EPKSDCKTHTCPPCP;
(f)
(SEQ ID NO: 6)
EPKSDKCTHTCPPCP;
(g)
(SEQ ID NO: 7)
EPKSDCKTHTVECPPCP;
(h)
(SEQ ID NO: 8)
EPKSDCKTVECPPCP;
(i)
(SEQ ID NO: 9)
EPKSDKCTHTVECPPCP;
(j)
(SEQ ID NO: 10)
EPPKSCDKTHTVECPPCP;
(k)
(SEQ ID NO: 11)
EPPKSDCKTHTVECPPCP;
(l)
(SEQ ID NO: 12)
EPPPKSCDKTHTVECPPCP;
(m)
(SEQ ID NO: 13)
EPPPPKSCDKTHTVECPPCP;
and
(n)
(SEQ ID NO: 28)
EPPKSDCKTKTVECPPCP.

7. The IgG antibody of any of claims 1-6, wherein the amino acid sequence comprised in the hinge is EPPKSDCKTKTVECPPCP (SEQ ID NO: 28).

8. The IgG antibody of any of claims 1-6, wherein each of the cysteine residue sandwiched between X₁ and X₂ on the two heavy chains form a disulfide bond therebetween.

9. The IgG antibody of any of claims 1-8, wherein the antibody further comprises a Fab domain that specifically binds to an antigen selected from the group consisting of EGFR, HER2, HER3, BCMA, B7H3, CEA, CEACAM6, claudin 18.2, c-MET, folate receptor, CD3, CD19, CD20, CD22, CD25, CD27L, CD30, CD33, CD37, CD48, CD56, CD70, CD73, CD74, CD79b, CD98, CD138, CD309 (VEGFR2), collagen IV, endothelin receptor ETB, ENPP3, fibronectin extra-domain B, GCC, GPNMB, LIV-1 (ZIP6), MUC1, MUC16, Mesothelin, NaPi2b, nectin 4, p-Cadherin, periostin, PSMA, SC-16 (anti-Fyn3), SLC44A4, SLTRK6, STEAP1, tenascin c, tissue factor, Trop2, and 5T4 (TPBG).

10. An isolated IgG antibody, comprising one of the following:
(a) two identical heavy chains each comprising an amino acid sequence of SEQ ID NO: 14; and two light chains each comprising an amino acid sequence of SEQ ID NO: 23;
(b) two identical heavy chains each comprising an amino acid sequence of SEQ ID NO: 15; and two light chains each comprising an amino acid sequence of SEQ ID NO: 23;
(c) two identical heavy chains each comprising an amino acid sequence of SEQ ID NO: 16; and two light chains each comprising an amino acid sequence of SEQ ID NO: 23;
(d) two identical heavy chains each comprising an amino acid sequence of SEQ ID NO: 17; and two light chains each comprising an amino acid sequence of SEQ ID NO: 23;
(e) two identical heavy chains each comprising an amino acid sequence of SEQ ID NO: 18; and two light chains each comprising an amino acid sequence of SEQ ID NO: 24;
(f) two identical heavy chains each comprising an amino acid sequence of SEQ ID NO: 19; and two light chains each comprising an amino acid sequence of SEQ ID NO: 25;
(g) two identical heavy chains each comprising an amino acid sequence of SEQ ID NO: 20; and two light chains each comprising an amino acid sequence of SEQ ID NO: 26;
(h) two identical heavy chains each comprising an amino acid sequence of SEQ ID ID: 21; and two light chains each comprising an amino acid sequence of SEQ TD NO: 25;
(i) two identical heavy chains each comprising an amino acid sequence of SEQ ID NO: 22, and two light chains each comprising an amino acid sequence of SEQ ID NO: 27;
(j) two identical heavy chains each comprising an amino acid sequence of SEQ ID NO: 29; and two light chains each comprising an amino acid sequence of SEQ ID NO: 31; and
(k) two identical heavy chains each comprising an amino acid sequence of SEQ ID NO: 30; and two light chains each comprising an amino acid sequence of SEQ ID NO: 32.

11. An antibody-drug conjugate (ADC) or a pharmaceutically acceptable salt thereof, comprising:
an antibody of any of the claims 1-10 conjugated to a cytotoxic drug by a chemical linker.

12. The ADC or the pharmaceutically acceptable salt thereof, of claim 9, wherein the cytotoxic drug is selected from the group consisting of monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, eribulin, ¹³¹I, interleukins, tumor necrosis factors, chemokines, irinotecan (SN38), exatecan, exatecan derivative, and nanoparticles.

13. The ADC or the pharmaceutically acceptable salt thereof, of any of claims 11-12, wherein the chemical linker comprises a portion that is selected from the group consisting of 6-maleimidocaproyl (MC), maleimidopropionyl (MP), valine-citrulline (Val-Cit), alanine-phenylalanine (Ala-Phe), p-aminobenzyloxycarbonyl (PAB), 6-maleimidocaproyl-valine-citrulline-p-aminobenzyloxycarbonyl (MC-Val-Cit-PAB), Mal-PEGₙ-Val-Cit-PAB (n=1-20), Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate.

14. The ADC or the pharmaceutically acceptable salt thereof, of any of claims 11-13, wherein each of the two heavy chains of the antibody comprises the amino acid sequence of SEQ ID NO: 21, each of the two light chains of the antibody comprises an amino acid sequence of SEQ ID NO: 25, and wherein the cytotoxic drug is eribulin or MMAE.

15. The ADC or the pharmaceutically acceptable salt thereof, of any of claims 11-13, wherein each of the two heavy chains of the antibody comprises the amino acid sequence of SEQ ID NO: 19, each of the two light chains of the antibody comprises an amino acid sequence of SEQ ID NO: 25, and wherein the cytotoxic drug is eribulin or MMAE.

16. The ADC or the pharmaceutically acceptable salt thereof, of any of claims 11-13, wherein each of the two heavy chains of the antibody comprises the amino acid sequence of SEQ ID NO: 20, each of the two light chains of the antibody comprises an amino acid sequence of SEQ ID NO: 26, and wherein the cytotoxic drug is eribulin or MMAE.

17. The ADC or the pharmaceutically acceptable salt thereof, of any of claims 11-13, wherein each of the two heavy chains of the antibody comprises the amino acid sequence of SEQ ID NO: 29, each of the two light chains of the antibody comprises an amino acid sequence of SEQ ID NO: 31, and wherein the cytotoxic drug is eribulin or MMAE.

18. The ADC or the pharmaceutically acceptable salt thereof, of any of claims 11-13, wherein each of the two heavy chains of the antibody comprises the amino acid sequence of SEQ ID NO: 30, each of the two light chains of the antibody comprises an amino acid sequence of SEQ ID NO: 32, and wherein the cytotoxic drug is eribulin or MMAE.

19. A preparation of the ADC or the pharmaceutically acceptable salt thereof, of any of claims 11-18, wherein at least 80% of the ADC are conjugates between the chemical linker with the antibody through the cysteines on the heavy chains of the antibody.

20. The preparation of the ADC or the pharmaceutically acceptable salt thereof, of any of claims 11-19, wherein ADC molecules having drug to antibody ratio (DAR) of 2 accounts for more than 60% of the total amount of ADC molecules.

21. An antibody-drug conjugate or a pharmaceutically acceptable salt thereof, comprising the reaction product of:
an antibody of any of the claims 1-10 which has undergone at least a partial reduction such that at least some H—H disulfide bonds between the corresponding cysteines in the hinge region of the antibody are reduced to free sulfhydryls; and
a chemical linker comprising a terminal thiol reactive group, attached to a cytotoxic drug molecule.

22. A method of producing a DAR2 antibody-drug conjugate (ADC) or a pharmaceutically acceptable salt thereof, comprising:
reducing the H—H disulfide bond between each of the cysteine residue sandwiched between X₁ and X₂ on the two heavy chains in the hinge region of the IgG antibody of any of claims 1-10 to obtain free sulfhydryls; and
reacting a chemical linker containing a terminal thiol reactive group to thereby conjugate one cytotoxic drug molecule to each of the two heavy chains of the antibody through the chemical linker.
